(19) United States
(12) Patent Application Publication
(10) Pub. No.: US 2022/0273119 A1
(43) Pub. Date: Sep. 1, 2022

(54) THERAPY CUSHION, APPARATUS AND METHOD FOR

(71) Applicant: LATEXFOAM TECHNOLOGY B.V., BN Winschoten (NL)

(72) Inventor: Jens Szymanska, BN Winschoten (NL)

(21) Appl. No.: 17/627,330

(22) PCT Filed: Jul. 15, 2020
(86) PCT No.: PCT/IB2020/056643

§ 371 Date: Jan. 14, 2022

(30) Foreign Application Priority Data
Jul. 15, 2019 (GB) 1910088.2

(51) Int. Cl. A47G 9/00; A61M 21/02; A47G 9/10; B29C 44/34; B29C 44/58

(52) U.S. Cl. CPC A47G 9/007; A61M 21/02; A47G 9/1045; B29C 44/3403; B29C 44/583; A61M 2021/0016

(57) ABSTRACT

Therapy cushion, comprising a cushion body, comprising a body part rest surface adapted to receive a human body part to be placed thereupon and comprising a first outlet opening therein and a first cavity being formed within the cushion body, wherein the first cavity is connected with the first outlet opening via a first outlet channel and wherein the first outlet opening is adapted such that a therapy compound stored in the first cavity can exit therethrough from the first cavity.

The present application relates to a therapy cushion, in particular an aromatherapy cushion, and an apparatus and a method to create such a therapy cushion.

It is an object of the present application to provide an improved therapy cushion and an improved apparatus and method for creating such a therapy cushion.

According to a first aspect, a therapy cushion is provided, comprising a cushion body, the cushion body comprising a body part rest surface adapted to receive a human body part to be placed thereupon and comprising a first outlet opening therein, the therapy cushion further comprising a first cavity being formed within the cushion body, wherein the first cavity is connected with the first outlet opening via a first outlet channel and wherein the first outlet opening is adapted such that a therapy compound stored in the first cavity can exit therethrough from the first cavity.

Therapy in the present disclosure is related to a treatment of the human body. A particular example of such a therapy in the present disclosure relates to aromatherapy. Aromatherapy is a therapy where aromatherapy compounds are delivered to a human user, in particular to a respiratory tract like the nose, mouth and lungs, to comfort the user. In particular, the therapy cushion is intended to provide a better and more relaxing rest and sleep experience to the user. Such an aromatherapy compound is for example an oil or another solution, such as an essential oil or an ethereal oil, containing natural fragrances like for example lavender lemongrass or the like.

Another particular form of therapy in the present disclosure is the repelling of insects, in particular such insects that feed on the human body. Examples for such insects that the present therapy cushion is capable to repel are flies, in particular mosquitos, that feed on the blood of humans. In this case the therapy compound is a mosquito repellant, in particular a natural mosquito repellant, such as, for example, an oil or another solution containing citronella, lemon eucalyptus or clove.

In particular, some compounds may have an application as a mosquito repellant and as an aromatherapy compound at the same time. Thus, they can be particularly well used in the therapy cushion.

The therapy cushion according to the present disclosure comprises a cushion body, wherein the cushion body comprises multiple surfaces, one of which is a body part rest surface, like, for example the top or upper surface when positioning the therapy cushion on a surface, like a bed frame or a couch. The body part rest surface is adapted such that at least a part of a human body part can be placed and rested thereupon, for example to rest or to sleep. The body part rest surface comprises a first outlet opening therein.

The cushion body is made from a flexible material, like, for example, Latex, which is a natural product. A method for producing such a therapy cushion will be described in detail below. The shape of the cushion body can be of any kind, like, for example, a substantially cuboid, ellipsoid, rotational ellipsoid or prism shape. The cushion body can, however, also be made of another flexible material, like, for example, a polyurethane foam or the like. The cushion may be of smaller dimensions, like, for example a width of 40 cm, a length of 40 cm and a height of 10 cm, to rest a human head, leg or arm thereupon. The cushion may also be of larger dimension, like, for example, a width of 80 cm, a length of 200 cm and a height of 20 cm, to rest the whole human body thereupon.

Further, the therapy cushion comprises at least a first cavity formed within the cushion body. The first cavity is fluid connected with the first outlet opening through a first outlet channel, which is formed within the cushion body. The first outlet channel has, for example, a substantially round or circular cross-sectional shape, wherein the outlet channel for example maintains the same or a similar cross-sectional shape from the first cavity to the first outlet opening. The first outlet opening, and the first outlet channel may have, for example, the same or a similar cross-sectional shape, in particular the same or a similar diameter.

The first outlet opening is adapted such that a therapy compound stored in the first cavity can exit therethrough from the first cavity. The therapy compound can be inserted, for example, through the first outlet opening and the first outlet channel into the first cavity.

The therapy cushion has the advantage, that a therapy compound can be easily deposited and released therefrom to comfort a human resting a body part, in particular his head, on the body part rest surface.

The therapy cushion can be improved in that the cushion body further comprises a first side surface comprising a first side opening therein; and wherein the first cavity is connected with the first side opening via a first insertion channel and wherein the first side opening is adapted such that a therapy compound can be inserted therethrough into the first cavity

As explained above, the cushion body may have multiple surfaces on multiple sides, one of which is the first side surface, which faces to a side of the therapy cushion, like, for example the left or right side thereof.

The first cavity is fluid connected with the first side opening through a first insertion channel, which is also formed within the therapy cushion. The first side opening, and the first insertion channel may have the same or a similar cross-sectional shape.

The first side opening is adapted such that a therapy compound can be inserted therethrough into the first cavity via the insertion channel and placed inside the first cavity.

This embodiment has the advantage that a therapy compound can be easily inserted through the side of the cushion body. Thereby, the first outlet opening can be designed much smaller, as the therapy compound can be inserted through the side opening.

The therapy cushion can be improved in that the first side opening is adapted such that the therapy compound stored in the first cavity cannot exit therethrough in a status of the therapy cushion where a human body part is resting on the body part rest surface.

This can be realized, for example, due to the shape of the cushion body and the arrangement of the first side opening therein. For example, the side opening is located afar from the center of the therapy cushion, such that a body part resting on the body part rest surface has no impact on a possible deformation of the side opening. Additionally, or alternatively, the first side opening is dimensioned in a way that no therapy compound can exit therethrough. Additionally, or alternatively, the insertion channel can be adapted such that the therapy compound stored in the first cavity cannot exit therethrough in a status where a human body part is resting on the body part rest surface.

This embodiment has the advantage that a user that rests a body part, and in particular his head, does not have to worry about the therapy compound exiting the “wrong” side of the therapy cushion but only through the first outlet opening.

The therapy cushion can be improved in that the first insertion channel and/or the first side opening is also adapted such that the therapy compound stored in the first cavity cannot exit therethrough in an unloaded status where no human body part is resting on the body part rest surface.

This embodiment has the advantage that the therapy compound can also not accidentally exit the therapy cushion when it is transported or is just placed on an even surface.

The therapy cushion can be improved in that the first outlet opening comprises a substantially half-moon-shaped cross section.

A substantially half-moon-shaped cross section can also be described as a curved or banana-shaped cross section. For example, the curved part of the cross section is curved and/or angled towards the center of the cushion, where a user would rest a body part, in particular his head.

This embodiment has the advantage that the therapy compound can exit the first cavity particularly well and reach the user where it has the most impact, i.e. the respiratory tract.

The therapy cushion can be improved in that it is further comprising a first locking plug that comprises a shape corresponding to the first outlet opening and that, when inserted into the first outlet opening, locks the outlet opening such that no therapy compound can exit therefrom.

The first locking plug can be substantially of the same material as the cushion body or from a different material. The first locking plug has substantially the same shape as the outlet opening and in particular as also at least a part of the first exit channel, such that it can be inserted therein at least in part. Thereby, the locking plug can lock the outlet opening such that no therapy compound can exit therefrom, if undesired.

This embodiment has the advantage that the therapy cushion can be transported very well without the therapy compound exiting from the first outlet opening and, in particular from the first cavity.

The therapy cushion can be improved in that the first side opening comprises a lower height than width.

In particular, the first side opening and/or the first insertion channel is designed in a way that the height is a factor higher than 2, 5 or 10 smaller than the width. For example, if the width of the first side opening and/or the first insertion channel is 5 cm wide, the height of the first side opening and/or the first insertion channel is 0.5 cm high.

This embodiment has the advantage that the therapy cushion can be transported very well without the therapy compound exiting from the first outlet opening and, in particular from the first cavity.

The therapy cushion can be improved in that the first cavity is substantially in the shape of an ellipsoid. Alternative shapes of the first cavity are substantially spherical or cuboid, with or without rounded edges and/or rounded corners.

This embodiment has the advantage that the therapy compound can be particularly well stored in the first cavity.

The therapy cushion can be improved in that the first outlet channel extends substantially vertically from the first cavity along a height of the cushion body.

This embodiment has the advantage that the therapy compound can reach the human body part, in particular a human head particularly well.

The therapy cushion can be improved in that the first outlet channel comprises of multiple first individual outlet channels that are arranged in parallel along a width of the cushion body.

In this embodiment, the multiple first individual outlet channels connect multiple first individual outlet openings with the first cavity.

This embodiment has the advantage that the therapy compound can reach individual parts of the human body part, in particular a human head, particularly well.

The therapy cushion can be improved in that the multiple first individual outlet channels and/or the first outlet channel are angled from the first cavity towards a center point of the cushion body.

This embodiment has the advantage that the therapy compound can reach the human body part, in particular the human head, particularly well.

The therapy cushion can be improved in that the surface of the first cavity is at least partially formed from a closed-pore material. In particular, all of the first cavity and the channels and openings formed within the cushion body are formed from a close pore material. In other words, all the surfaces of the inner holes formed within or on the cushion body have a close-pore structure.

As described above, the cushion body is made for example, from Latex. By using Latex material and applying an electromagnetic frequency radiation during curing of the Latex material, the Latex forms a close-pore surface against cores being arranged as placeholders for the first cavity and/or the channels and openings in the uncured latex foam. This will be described in further detail below.

This effect cannot be achieved by forming the first cavity and/or the channels and openings into the cushion body through a machining or cutting method when using Latex material.

This embodiment has the advantage that the therapy compound cannot leak into open pores and dissolve or remain therein without reaching the human body.

The therapy cushion can already comprise the therapy compound, in particular the aromatherapy compound, in the cavity. In this way the therapy cushion is ready to use.

However, the therapy cushion can also be provided without the therapy compound. In this way, the therapy compound can be carefully selected by the user of the therapy cushion and placed in the cavity himself.

It goes without saying that the therapy compound can be replaced, in particular through the side opening, once the therapy compound originally or previously placed in the cavity has worn off or is of no longer use.

According to another aspect, an apparatus for creating a therapy cushion, in particular a therapy cushion according one of the embodiments as described above, is provided.

The apparatus comprises an upper mold half, the upper mold half comprising a sprue, and a lower mold half. The apparatus further comprises a first core, the first core comprising a first vertical core holder and a first center core piece attached to one end of the first vertical core holder. The first vertical core holder is on another end attached to a bottom of the lower mold half and extends therefrom at least partially into a space being formed between the upper mold half and the lower mold half and positioning the center core piece therebetween.

The apparatus can be improved in that if further comprises an electromagnetic frequency generator that is adapted to apply a frequency of approximately 27 MHz and a voltage between 5000 V to 20000 V in a distance between 10 cm to 20 cm to a Latex foam being positioned inside the space between the upper mold half and the lower mold half

The apparatus can be improved in that the first core further comprises a first lateral core holder being attached to a side of the lower mold half and/or the upper mold half and to the first center core piece.

The apparatus can be improved in that the first lateral holder is being fixed between the upper mold half and the lower mold half in their respective side walls.

According to another aspect, a method for creating a therapy cushion, in particular a therapy cushion according one of the embodiments as described above, is provided. The method can particularly well be performed with an apparatus for creating a therapy cushion according to one of the embodiments as described above.

The method comprises the steps of providing a lower mold half, inserting and attaching a first core to the bottom of the lower mold half, the first core comprising a first vertical core holder and a first center core piece attached to one end of the first vertical core holder, attaching an upper mold half on top of the lower mold half such that a space is being formed therebetween, the upper mold half comprising a sprue, introducing Latex foam through the sprue into the space formed between the lower mold half and the upper mold half, curing the Latex foam, removing the upper mold half, removing the cured Latex from the lower mold, extracting the first core from the cured Latex.

The method can be improved in that the step of curing the Latex is achieved by applying an electromagnetic frequency with a frequency of approximately 27 MHz and a voltage between 5000 V to 20000 V in a distance between 10 cm to 20 cm to the Latex foam.

The method can be improved in that the step of inserting and attaching a first core to the bottom of the lower mold half comprises attaching the first vertical core holder to the bottom of the lower mold half.

The method can be improved in that the first core further comprises a first lateral core holder and in that the method further comprises fixing the first lateral core holder between the upper mold half and the lower mold half in their respective side walls.

The method can be improved in that, before inserting and attaching the first and/or second core and/or parts thereof to the lower mold half, coating the first and/or second core and/or parts thereof with Latex material and air-curing the coating. In that way, a close-pore surface of the cavity, opening and/or channel can be achieved. When curing the Latex, in particular with the electromagnetic frequency, the coating merges with the remaining Latex in the mold halves, thus creating a sealed surface.

Embodiments will now be described in further detail with reference to the following figures in which

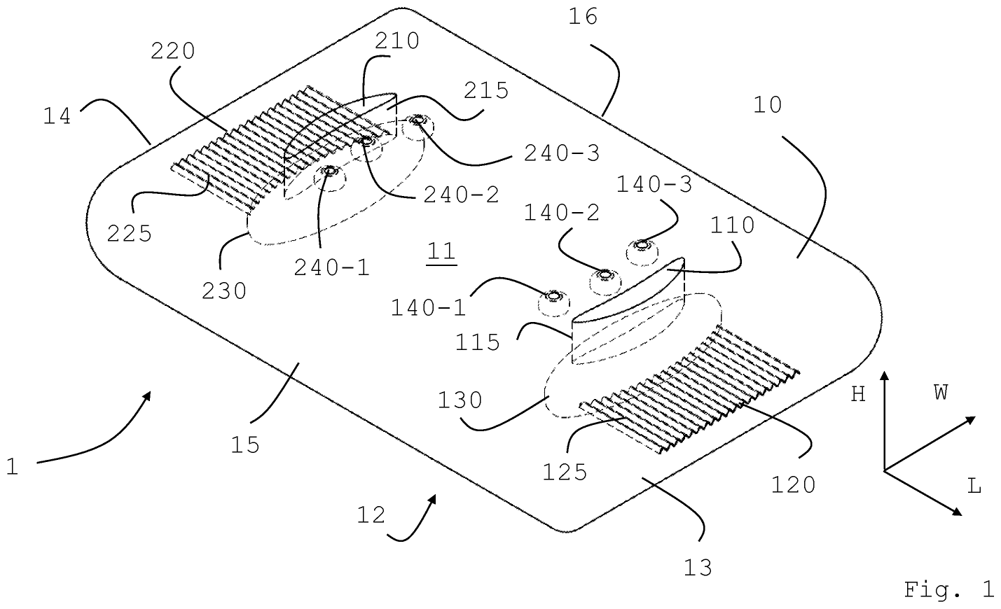

FIG. 1 is a schematic perspective view of a therapy cushion 1 according to an embodiment,

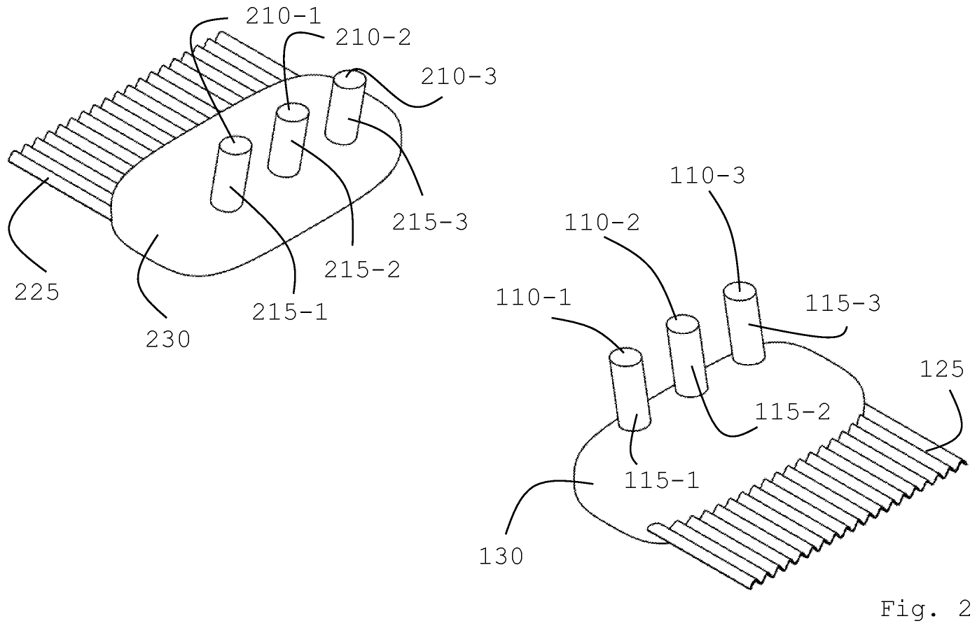

FIG. 2 is a schematic perspective view of an alternative embodiment of the therapy cushion 1 of FIG. 1,

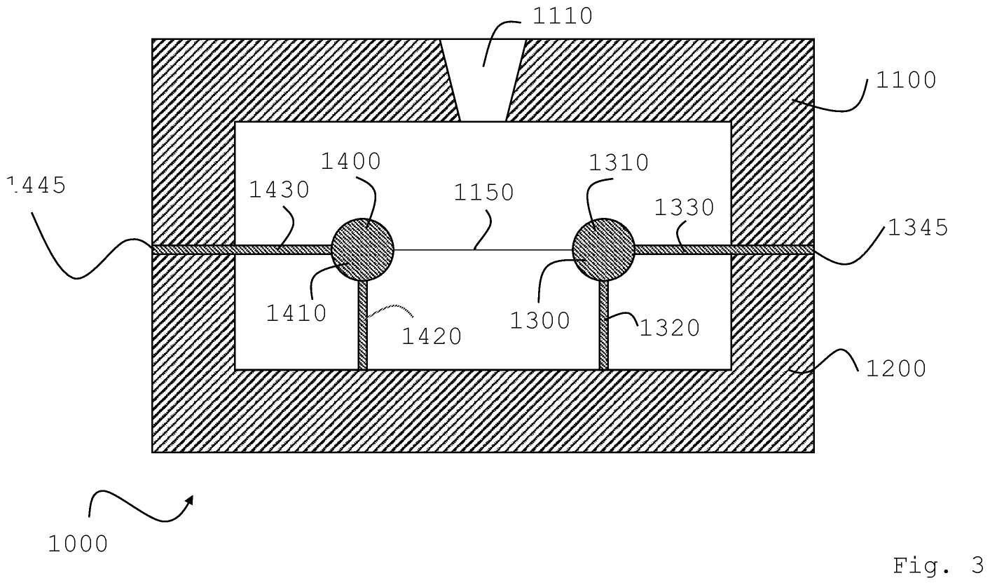

FIG. 3 is a schematic cross-sectional view of an apparatus according to and embodiment, and

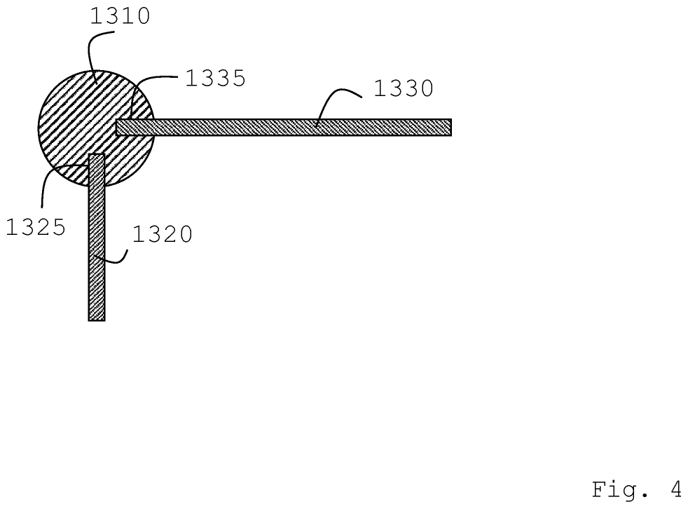

FIG. 4 is a detailed schematic cross-sectional view of an aspect of the apparatus according to an embodiment.

In the figures, the same reference numeral refers to the same or a similar feature.

FIG. 1 depicts a schematic perspective view of a therapy cushion 1 according to an embodiment. The therapy cushion 1 comprises a three-dimensional cushion body 10, which is exemplarily depicted as a cuboid 10 with rounded edges and rounded corners. The therapy cushion has an extension in height H, width W and length L. As can be seen in FIG. 1 the therapy cushion 1 is substantially symmetric with regard to a central axis along the width W and the length L of the cushion.

The cushion body 10 comprises six surfaces, an upper or top surface 11, which is named the body part rest surface 11, a bottom or lower surface 12, a first, right side surface 13, a second, left side surface 14, a front side surface 15 and a back side surface 16. The body part rest surface 11 is adapted and arranged to receive a human body part to be placed on the therapy cushion 1, for example for resting or sleeping. The cushion body 10 is essentially made of Latex, which is a natural and flexible material, and has multiple cavities and channels formed therein, which are described below in detail.

There are two openings in the body part rest surface, a first, right outlet opening 110 and a second, left outlet opening 210. There are two openings in the side surfaces, a first, right side opening 120 and a second, left side opening 220. Inside the cushion body 10 there are two cavities formed, a first, right cavity 130 and a second, left cavity 230. Further, the therapy cushion 1 comprises a first, right insertion channel 125 and a second, left insertion channel 225. Additionally, the therapy cushion 1 comprises a first, right outlet channel 115 and a second, left outlet channel 215.

The first insertion channel 125 connects the first side opening 120 and thereby the exterior of the therapy cushion 1 with the first cavity 130. In the same manner, the second insertion channel 225 connects the second side opening 220 and thereby the exterior of the therapy cushion 1 with the second cavity 230. Additionally, the first outlet channel 115 connects the first cavity 130 with the first outlet opening 110 and thereby the exterior of the therapy cushion 1 and the second outlet channel 215 connects the second cavity 230 with the second outlet opening 210 and thereby the exterior of the therapy cushion 1.

The first side opening 120 is adapted such that a therapy compound can be inserted therethrough into the first cavity 130 via the first insertion channel 125 and, likewise, the second side opening 220 is adapted such that a therapy compound can be inserted therethrough into the second cavity 230 via the second insertion channel 225.

However, in an unloaded position and when a human body part, in particular a head, is resting on the therapy cushion 1, the first side opening 120 and the second side opening 220 and at least a part of the first insertion channel 125 and the second insertion channel 225 are shut and closed, such that in such a scenario, i.e. when the therapy cushion 1 is lying on an even surface and if a human body part, in particular a head, is resting on the therapy cushion 1, essentially none of the therapy compound being inserted into the first cavity 130 and the second cavity 230 can exit through one of the side openings.

The first outlet opening 110 is adapted such that the therapy compound stored in the first cavity 130 can exit therethrough to the exterior of the therapy cushion 1 via the first outlet channel 115. Likewise, the second outlet opening 210 is adapted such that the therapy compound stored in the second cavity 230 can exit therethrough to the exterior of the therapy cushion 1 via the second outlet channel 215. In contrast to the side openings, the outlet openings are particularly open and unclosed if a body part rests on the therapy cushion 1.

The first cavity 130 and the second cavity 230 are both formed within the cushion body 10 and form a space that is depicted as being essentially in the form of an ellipsoid and that is adapted to receive and store a therapy compound that is inserted therein through the respective side openings and insertion channels, as described above. Other forms of the first and second cavity are however possible, like, for example, an essentially spheroidal form.

As can be seen in FIG. 1, the first insertion channel 125 and the second insertion channel 225 extend substantially horizontally along the length and centrally with regard to the height H from the respective side openings to the center of the therapy cushion 1 until they terminate into the respective first and second cavity at a center with regard to the height of the respective cavity. The first cavity 130 and the second cavity 230 are both formed within the cushion body 10 and are arranged essentially centered with regard to the height H and the width W of the therapy cushion 1.

As the first cavity 130 and the second cavity 230 both have longer extension in height H, the cavity is larger with respect to the upper side and the lower side of the cushion body 10 as the first insertion channel 125 and the second insertion channel 225. This facilitates that a therapy compound stored in the first and second cavity cannot exit through the first and second insertion channel and the first and second side opening, respectively, when the therapy cushion is placed on a horizontal surface in rest or when a body part rests on the body part rest surface.

Further, the first insertion channel 125 and the second insertion channel 225 have essentially the same dimension in width W and height H as their respective side openings and both have a much smaller height H in comparison to their width W. In particular, the height of the first side opening 120 and the second side opening 220 and also the first insertion channel 125 and the second insertion channel 225 is less than 1 mm or even zero when in rest or under load. In other words, the respective openings and channels are flush and fully closed. This also facilitates that a therapy compound stored in the first and second cavity cannot exit through the first and second insertion channel and the first and second side opening, respectively, when the therapy cushion is placed on a horizontal surface in rest or when a body part rests on the rest surface.

The first outlet channel 115 and the second outlet channel 215 have essentially the same dimensions in width W and length L as their respective outlet openings and a shape that is inwardly curved to the center point of the therapy cushion 1 with regard to their respective length L and width W. Alternatively, the shape of the first and second outlet opening can be described as a half moon-shape or banana-shape, which is bent towards the center point of the therapy cushion 1.

The first outlet channel 110 and the second outlet channel 210 extend substantially vertically from the respective cavity. They also extend substantially vertical with regard to a center pane in the directions of width W and length L through the therapy cushion 1 and also to the body part rest surface 11. Similarly, the respective cross-section of the first outlet opening 110 and the second outlet opening 210 are substantially parallel to the center pane in the directions of width W and length L through the therapy cushion 1 and also parallel to the body part rest surface 11.

The first and second outlet openings are positioned in the body part rest surface such that they would be directly next or in close proximity to a human body part, in particular a head, being placed centrally on the therapy cushion 1. Thereby, the therapy compounds can exit right next to the body part and unfold its full therapeutic effect.

Further, the therapy cushion 1 may comprise one or more gemstones. These gemstones can be provided in particular in the body part rest surface 11. As an example, there are three gemstones provided on the respective inner side of each of the first and second outlet opening 110 and 210. The three exemplary gemstones 140-1, 140-2 and 140-3 are placed in a half-moon fashion, slightly curved next to the first outlet opening 110. Similarly, the other three exemplary gemstones 240-1, 240-2 and 240-3 are placed in the same fashion next to the second outlet opening 210. In particular, gemstones can have a healing effect on the human body. Thus, any kind of therapy being conducted or provided by the therapy cushion 1 is being supported by the provision of gemstones.

All surfaces of the cavities, channels and openings are in particular closed-pore surfaces. This can for example be achieved by that any cores being used as placeholders to form the cavities, channels and openings in the therapy cushion are coated with a coating, for example a Latex coating, and letting that coating dry out prior to using the cores to form the cavities, channels and openings, as will be described in more detail below in relation to FIGS. 3 and 4.

FIG. 2 is a schematic perspective view of an alternative embodiment of the therapy cushion 1 of FIG. 1, wherein the cushion body has been omitted for visibility reasons. In other words, FIG. 2 only shows the hollow spaced being formed in the cushion body, which may have the same or a similar shape as shown in FIG. 1.

In contrast to the embodiment as shown in FIG. 1, the first outlet channel comprises of multiple first individual outlet channels 115-1, 115-2 and 115-3, each of which connects a first individual outlet opening 110-1, 110-2 and 110-3 with the first cavity.

Similarly, the second outlet channel comprises of multiple second individual outlet channels 215-1, 215-2 and 215-3, each of which connects a first individual outlet opening 210-1, 210-2 and 210-3 with the first cavity. The multiple first and second individual outlet channels are in this embodiment angled towards a center point of the cushion body.

FIG. 3 shows an apparatus 1000 according to an embodiment for creating a therapy cushion, in particular a therapy cushion according one of the embodiments as described above.

The apparatus 1000 comprises an upper mold half 1100, the upper mold half 1100 comprising a sprue 1110, and a lower mold half 1200. The apparatus 1000 further comprises a first core 1300, the first core 1300 comprising a first vertical core holder 1320 and a first center core piece 1310 attached to one end of the first vertical core holder 1320. The first vertical core holder 1320 is on another end attached to a bottom of the lower mold half 1200 and extends therefrom at least partially into a space being formed between the upper mold half 1100 and the lower mold half 1200 and positioning the first center core piece 1310 therebetween. Further, the first core 1300 comprises a first lateral core holder 1330 being attached to the first center core piece 1310 and being fixed between the upper mold half 1100 and the lower mold half 1200 in their respective side walls with a first mold insertion part 1345 of the first lateral core holder 1330 along a centerline 1150.

Similarly, the apparatus 1000 comprises a second core 1400, the second core 1400 comprising a second vertical core holder 1420 and a second center core piece 1410 attached to one end of the second vertical core holder 1420. The second vertical core holder 1420 is on another end attached to a bottom of the lower mold half 1200 and extends therefrom at least partially into a space being formed between the upper mold half 1100 and the lower mold half 1200 and positioning the second center core piece 1410 therebetween. Further, the second core 1400 comprises a second lateral core holder 1430 being attached to the second center core piece 1410 and being fixed between the upper mold half 1100 and the lower mold half 1200 in their respective side walls with a second mold insertion part 1445 of the second lateral core holder 1430 along a centerline 1150.

The apparatus further comprises an electromagnetic frequency generator (not shown) that is adapted to apply a frequency of approximately 27 MHz and a voltage between 5000 V to 20000 V in a distance between 10 cm to 20 cm to a Latex foam being positioned inside the space between the upper mold half 1100 and the lower mold half 1200.

FIG. 4 shows a detail of the first core as shown in FIG. 3. Therein, the first lateral core holder 1330 is depicted being inserted into the first center core piece 1310 with a first core insertion part 1335 of the lateral core holder 1330. Similarly, the first vertical core holder 1320 is shown as being inserted into the first center core piece 1310 with a first core insertion part 1325 of the first vertical core holder 1320.

By inserting respective parts of the lateral and vertical core holder, it is possible, after curation of the cushion, to easily remove the lateral and vertical core holders to consecutively remove the therapy cushion.

The first core 1300, the second core 1400, either as a whole or only parts thereof, in particular the first center core piece 1310, the first vertical core holder 1320, the first core insertion part of vertical holder 1325, the first lateral core holder 1330, the second center core piece 1410, the second vertical core holder 1420, the second lateral core holder 1430 may be coated with Latex material before using the respective cores or core parts before inserting and attaching the respective core or core parts to the mold half.

### REFERENCE NUMERALS

1 therapy cushion
10 cushion body
11 body part rest surface
12 bottom surface
13 first side surface
14 second side surface
15 front side surface
16 back side surface
110 first outlet opening
110-1 first individual outlet opening
110-2 first individual outlet opening
110-3 first individual outlet opening
115 first outlet channel
115-1 first individual outlet channel
115-2 first individual outlet channel
115-3 first individual outlet channel
120 first side opening
125 first insertion channel
130 first cavity
140-1 gemstone
140-2 gemstone
140-3 gemstone
240-1 gemstone
240-2 gemstone
240-3 gemstone
210 second outlet opening
210-1 second individual outlet opening
210-2 second individual outlet opening
210-3 second individual outlet opening
215 second outlet channel
215-1 second individual outlet channel
215-2 second individual outlet channel
215-3 second individual outlet channel
220 second side opening
225 second insertion channel
230 second cavity
1000 apparatus for creating a therapy cushion
1100 upper mold half
1110 sprue
1150 centerline
1200 lower mold half
1300 first core
1310 first center core piece
1320 first vertical core holder
1325 first core insertion part of vertical holder
1330 first lateral core holder
1335 first core insertion part of first lateral core holder
1345 first mold insertion part of first lateral core holder
1400 second core
1410 second center core piece
1420 second vertical core holder
1430 second lateral core holder
1445 second mold insertion part of second lateral core holder
W width
H height
L length

1.-19. (canceled)

20. A therapy cushion comprising:
a cushion body comprising a body part rest surface adapted to receive a human body part to be placed thereupon and comprising a first outlet opening therein and a first cavity being formed within the cushion body, wherein the first cavity is connected with the first outlet opening via a first outlet channel and wherein the first outlet opening is adapted such that a therapy compound stored in the first cavity can exit therethrough from the first cavity.

21. The therapy cushion according to claim 20, wherein the cushion body further comprises a first side surface comprising a first side opening therein and wherein the first cavity is connected with the first side opening via a first insertion channel and wherein the first side opening is adapted such that a therapy compound can be inserted therethrough into the first cavity.

22. The therapy cushion according to claim 21, wherein the first side opening is adapted such that the therapy compound stored in the first cavity cannot exit therethrough in a status of the therapy cushion where a human body part is resting on the body part rest surface.

23. The therapy cushion according to claim 20, wherein the first outlet opening comprises a substantially half-moon-shaped cross section.

24. The therapy cushion according to claim 20, further comprising a first locking plug that comprises a shape corresponding to the first outlet opening and that, when inserted into the first outlet opening, locks the outlet opening such that no therapy compound can exit therefrom.

25. The therapy cushion according to claim 21, wherein the first side opening comprises a lower height than width.

26. The therapy cushion according to claim 20, wherein the first cavity is substantially in the shape of an ellipsoid.

27. The therapy cushion according to claim 20, wherein the first outlet channel extends substantially vertically from the first cavity along a height of the cushion body.

28. The therapy cushion according to claim 20, wherein the first outlet channel comprises of multiple first individual outlet channels that are arranged in parallel along a width of the cushion body.

29. The therapy cushion according to claim 28, wherein the multiple first individual outlet channels are angled from the first cavity towards a center point of the cushion body.

30. The therapy cushion according to claim 20, wherein the surface of the first cavity is at least partially formed from a closed-pore material.

31. An apparatus for creating a therapy cushion, the apparatus comprising:
an upper mold half, the upper mold half comprising a sprue,
a lower mold half,
a first core, the first core comprising a first vertical core holder and a first center core piece attached to one end of the first vertical core holder, the first vertical core holder being on another end attached to a bottom of the lower mold half and extends therefrom at least partially into a space being formed between the upper mold half and the lower mold half and positioning the center core piece therebetween.

32. The apparatus according to claim 31, further comprising an electromagnetic frequency generator that is adapted to apply a frequency of approximately 27 MHz and a voltage between 5000 V to 20000 V in a distance between 10 cm to 20 cm to a Latex foam being positioned inside the space between the upper mold half and the lower mold half.

33. The apparatus according to claim 31, wherein the first core further comprises a first lateral core holder being attached to a side of the lower mold half and/or the upper mold half and to the first center core piece.

34. The apparatus according to claim 31, wherein the first lateral holder is being fixed between the upper mold half and the lower mold half in their respective side walls.

35. A method for creating a therapy cushion, the method comprising: providing a lower mold half,
inserting and attaching a first core to the bottom of the lower mold half, the first core comprising a first vertical core holder and a first center core piece attached to one end of the first vertical core holder,
attaching an upper mold half on top of the lower mold half such that a space is being formed therebetween, the upper mold half comprising a sprue,
introducing Latex foam through the sprue into the space formed between the lower mold half and the upper mold half,
curing the Latex foam,
removing the upper mold half,
removing the cured Latex from the lower mold, and
extracting the first core from the cured Latex.

36. The method according to claim 35, wherein the step of curing the Latex is achieved by applying an electromagnetic frequency with a frequency of approximately 27 MHz and a voltage between 5000 V to 20000 V in a distance between 10 cm to 20 cm to the Latex foam.

37. The method according to claim 35, wherein the step of inserting and attaching a first core to the bottom of the lower mold half comprises attaching the first vertical core holder to the bottom of the lower mold half.

38. The method according to claim 35, wherein the first core further comprises a first lateral core holder and wherein the method further comprises the step of fixing the first lateral core holder between the upper mold half and the lower mold half in their respective side walls.
